(19) United States
(12) Patent Application Publication
(10) Pub. No.: US 2024/0189569 A1
(43) Pub. Date: Jun. 13, 2024

(54) VENT DEVICES AND RELATED METHODS

(71) Applicant: B. Braun Melsungen AG, Melsungen (DE)

(72) Inventor: Khai Tick Sim, Penang (MY)

(73) Assignee: B. Braun Melsungen AG, Melsungen (DE)

(21) Appl. No.: 18/555,097

(22) PCT Filed: Apr. 11, 2022
(86) PCT No.: PCT/EP2022/059640

§ 371 Date: Oct. 12, 2023

Related U.S. Application Data
(60) Provisional application No. 63/173,855, filed on Apr. 12, 2021.

(51) Int. Cl. A61M 39/26

(52) U.S. Cl. CPC A61M 39/26; A61M 2039/266; A61M 2205/02

(57) ABSTRACT

A vent device (100, 140) for venting gas from a line of a medical device having gas to be removed has several different variations. The vent device can include an inlet (102) in communication, when in use, with an interior of the line, and an outlet (104) in communication with air outside the line or is open to the atmosphere. The inlet and the outlet define opposite ends of a flow path (106) configured to vent gas from the interior of the line out to the outside of the line, and to inhibit flow of liquid from the interior of the line out the outlet of the vent device.

### TECHNICAL FIELD

The present invention relates, in general terms, to a vent device for venting gas from a medical device, such as a tubing line. In some cases, the present invention can be used to vent gas from a peripheral intravenous catheter, or other medical devices.

### BACKGROUND

A venous air embolism occurs when one or more bubbles enter a vein and restrict blood supply to particular human organs such as the heart, a lung, the brain, and so on. Arterial air embolisms are the same event, though occurring in an artery.

Air embolisms can cause a heart attack, stroke, respiratory failure, and fatality especially in neonatal patients wherein 0.02 mL of air can cause a neonatal patient to suffer from ischemia or a restriction in blood supply to tissue.

Air embolisms can occur as a result of compressive injury, lung trauma, and compression such as through scuba diving, but also through injections and surgical procedures, which can occur during brain surgery and infusion therapy where air trapped in the infusion line is not properly evacuated before drug infusion.

Currently, medical practitioners are trained to identify air embolism and remove it. This is responsive action rather than pre-emptive action and therefore less desirable. In other cases, practitioners expel air from the infusion line using a syringe. Practitioners can aspirate trapped air by connecting a syringe to a needleless connector. This requires the practitioner to identify the trapped air and then remove it all.

The above-noted remedies require a practitioner to identify an issue and resolve it, rather than removing or reducing the likelihood of the issue occurring.

Current air aspiration techniques result in additional time due to additional steps needing to be performed. This is undesirable in emergency where practitioners may already be distracted by other symptoms and treatments. Moreover, there is additional waste and cost resulting from syringe use and the practitioner's time.

It would be desirable to overcome or ameliorate at least one of the above-described problems, or at least provide a useful alternative.

### SUMMARY

Disclosed is a vent device for venting gas from a line, the device comprising: an inlet in communication, in use, with an interior of the line; and an outlet in communication with air outside the line; wherein the inlet and outlet define opposite ends of a flow path configured to vent gas from the interior of the line out the outlet, and to inhibit flow of liquid from the interior of the line out the outlet.

The flow path may comprise a gas-permeable, hydrophobic material.

The device may further comprise a housing, the inlet being at a distal region of the housing, the outlet being at a proximal region of the housing, and the flow path forming a conduit within the housing. The outlet may comprise the hydrophobic material. The housing may comprise one or more proximal outlet apertures and the outlet may then be formed as a strip of hydrophobic material across the one or more outlet apertures. The strip may cover the one or more outlet apertures.

The hydrophobic material may be embodied as an insert and located in the housing, disposed between the inlet and outlet.

The inlet may comprise one or more inlet apertures formed in the distal region of the housing.

The flow path may be formed by a body of the hydrophobic material. The outlet may be defined by a proximal region of the body and the inlet may then be defined by a distal region of the body.

The device may comprise a distal region at least partially comprising the inlet, a proximal region comprising the outlet and an intermediate region intermediate the distal region and proximal region. The distal region may have a first thickness and the intermediate region may have a second thickness that is smaller than the first thickness. The proximal region may have a third thickness that is greater than the second thickness.

The proximal region may be configured as a grip. An external surface of the proximal region may be textured. The texturing increases friction between the device a finger of a user.

The distal region may have a first width and the proximal region may then have a second width that is greater than the first width.

The line may be part of an intravenous catheter (IVC) or peripheral intravenous catheter (PIVC). The vent device may be sized to be received through a needleless connector in the IVC or PIVC. In this regard, being sized to be received through the needleless connector may involve only that portion which is inserted into or through the needleless connector being so sized. Any portion of the device that has no need to be inserted may be of an appropriate size, which can be configured to be gripped by the user.

Advantageously, the device, when connected to the line having gas to be removed, such as through a needleless connector, may passively allow the passage of air along the flow path, from the device, to the ambient surrounding. Moreover, since the flow path, which can include a hydrophobic material, is configured to inhibit the passage of liquid out the outlet, there is little or no risk of blood leakage from the device or through the connector by which the device connects to the line.

A vent device for venting gas from a medical device is disclosed. The vent device can comprise: an inlet in communication, in use, with an interior of the medical device; and an outlet open to air outside of the medical device; wherein the inlet and the outlet define opposite ends of a flow path configured to vent gas from the interior of the medical device out the outlet and inhibit flow of liquid from the interior of the medical device out the outlet.

The vent device of claim 1, wherein the flow path comprises a gas-permeable hydrophobic material.

The vent device can further include a housing, the inlet being at a distal region of the housing, the outlet being at a proximal region of the housing, and the flow path forming a conduit within the housing. For example, the housing can be a two-part housing with a seam and wherein an insert is located in a cavity of the housing to define the vent device.

The hydrophobic material can be located at the outlet.

The housing can comprise one or more proximal outlet apertures and the hydrophobic material can be located at the outlet or a strip of hydrophobic material can be positioned across the one or more outlet apertures.

The strip of hydrophobic material can cover the one or more outlet apertures such that gas flowing out of the one or more apertures must flow through the strip of hydrophobic material.

The hydrophobic material can be formed as an insert and the insert can be located in a cavity of the housing and disposed between the inlet and outlet.

The inlet can comprise one or more inlet apertures formed in the distal region of the housing.

The flow path can be formed by a body of the hydrophobic material.

The vent device can have a generally flat profile with a length and a thickness, and wherein the length is at least 20 times greater than that of the thickness. For example, the length can be 25 times greater, 30 times greater, 35 times greater, 40 times greater, or 60 times greater than the thickness.

The vent device can comprise a distal region at least partially comprising the inlet, a proximal region comprising the outlet and an intermediate region intermediate the distal region and proximal region.

The distal region can have a first thickness and the intermediate region can have a second thickness that is smaller than the first thickness.

The proximal region can have a third thickness that is greater than the second thickness. The proximal region of the vent device can be configured as a grip.

An external surface of the proximal region can be textured to improve gripping.

The distal region can have a first width and the proximal region can have a second width that is greater than the first width.

The line can be part of an intravenous catheter (IVC) or peripheral intravenous catheter (PIVC).

The inlet can be located within a receiving end of a port or a connector.

When in use, gas vents through the vent device without actuating a first part of the vent device relative to a second part of the vent device, which does not incorporate first and second parts that are movable relative to one another for actuation.

A method of forming a vent device for venting gas from a line having gas to be removed is disclosed. The method can comprise: forming a body from a hydrophobic material with a distal region, a proximal region, and intermediate region between the distal region and the proximal region; providing the body with a length that is at least 20 times greater than a thickness of the body to form a thin profile; forming the distal region with a first width W₁ and the proximal region with a second width W₂ with the second width W₂ being greater than the first width W₁; forming the distal region with a first thickness H₁, the intermediate region with a second thickness H₂, and the proximal region with a third thickness H₃; and providing a tapered edge at the distal region to facilitate insertion of the vent device.

The first thickness H₁ can be greater than the second thickness H₂.

### BRIEF DESCRIPTION OF THE DRAWINGS

Embodiments of the present invention will now be described, by way of non-limiting examples, with reference to the drawings in which:

FIG. 1 is a rear perspective view of a device according to an embodiment of the present teachings;

FIG. 2 is a front perspective view of the device;

FIG. 3 is a side view of the device;

FIG. 4 another perspective view of the device;

FIG. 5 is a side elevation view of the device;

FIG. 6 is a top plan view of the device;

FIG. 7 is an exploded view of the device showing a two part housing and an insert;

FIG. 8 is a cross-section end view of the device of FIG. 7, in an assembled state;

FIG. 9 is a device according to an embodiment of the present teachings with a grip on a proximal region of the device, with different texture options;

FIGS. 10 and 11 are a front perspective and a rear perspective view, respectively, of an alternative device according to an embodiment of the present teachings;

FIG. 12 is a front perspective view of an alternative device according to an embodiment of the present teachings;

FIG. 13 shows a PIVC prior to connection to the vasculature of a patient;

FIG. 14 shows the PIVC assembly of FIG. 13 after connection to the vasculature of the patient;

FIG. 15 shows a device according to an embodiment of the present teachings, inserted through a port or connector attached to the PIVC and into fluid communication with the PIVC;

FIG. 16 shows a device according to an embodiment of the present teachings in connection with a housing of a connector for accessing a line, to vent gas from the line; and

FIG. 17 shows the connector or valve contact area of a device according to an embodiment of the present teachings, and the manner in which that device is received in the connector or valve.

### DETAILED DESCRIPTION

The detailed description set forth below in connection with the appended drawings is intended as a description of the presently preferred embodiments of vent devices provided in accordance with aspects of the present devices, systems, and methods and is not intended to represent the only forms in which the present device, system, and method may be constructed or utilized. The description sets forth the features and the steps for constructing and using the embodiments of the present device, system, and method in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

Generally speaking, practitioners must identify trapped air in infusion set, catheter systems and the like or, after delivery of air into a patient, the presence of an embolism and ameliorate it. However, emergency situations place pressure on practitioners who may be distracted by other matters and not identify trapped air prior to its delivery into a patient. Embodiments of the present invention enable passive venting of air from a line of a medical device, such as a peripheral intravenous catheter (PIVC) or an intravenous catheter (IVC), without particularly inspecting for trapped air and intervening by a practitioner. Embodiments of the invention can also prevent blood and liquid from following the flow path of the air and leaking from the vent device.

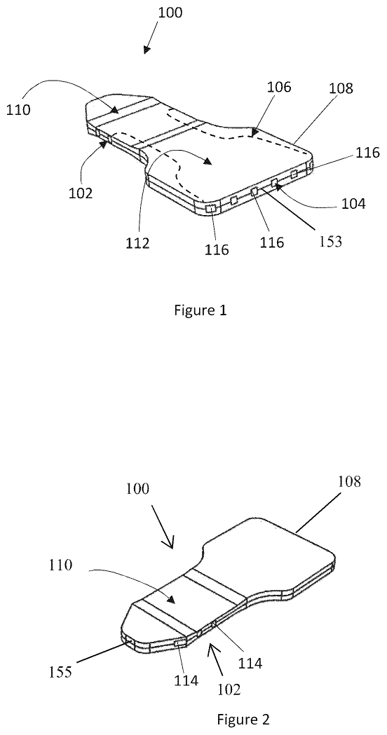

With reference now to FIG. 1, a vent or venting device 100 is illustrated comprising an inlet 102, an outlet 104, and a flow path 106 between the inlet and the outlet. In other words, the flow path 106 has an inlet and an outlet at opposite ends of the flow path.

In the embodiment shown in FIG. 1, the vent or venting device 100, or simply device for short, includes a housing 108. The inlet 102 is at a distal region 110 of the housing 108 and the outlet 104 is at a proximal region 112 of the housing 108. The flow path 106 (illustrated in broken lines as it is internal of the device 100) thus forms a conduit within the housing 108.

The distal region 110 of the housing 108 can be the part of the housing 108 that is in communication with gas (e.g., air) in a line, system, or chamber of a medical device during use of the vent device 100. The shape of the device 100 is not limited to the shape shown and can be adjusted as needed. For example, the distal region 110 can be whatever portion of the housing 108 is in communication with gas in the line. As shown, the housing 108 has a flat profile in that the width of the housing is substantially larger or greater in dimension than the thickness of the housing. The flat profile allows the housing 108 to be inserted into a body of a connector having a complementary narrow opening profile. In other examples, the housing 108 can have a distal region having a cylindrical-like shape for insertion into a complementary bore-like inlet opening. For example, the housing 108 of the vent device 100 can have male Luer tip for inserting into a female Luer of a connector to vent air out from the connector side and out of the vent device.

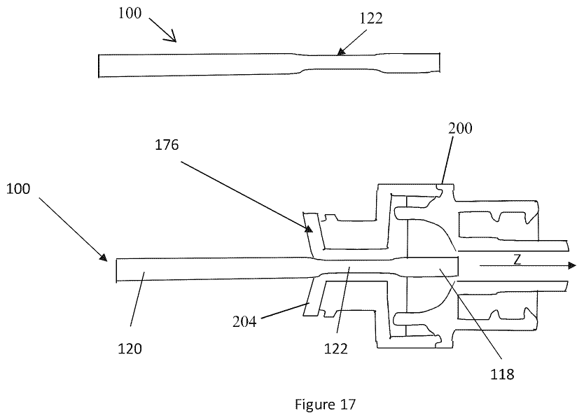

When the vent device 100 is in use to remove gas from a medical device, such as from a line that is part of an IVC or PIVC, the inlet 102 is in communication with the interior of the line, such as shown in FIGS. 16 and 17. The vent device 100 may be supplied separately from the line, system, or assembly of the medical device, or may be provided, in situ, with the medical device, such as during packaging of the medical device, so as to enable rapid passive use of the vent device 100 without a separate connection step.

With reference to FIG. 2, the inlet 102 is presently configured as a plurality of apertures 114 only some of which, for clarity of FIG. 2, are provided with reference numerals. The apertures 114 are formed at the distal region 110 of the housing 108. It will be appreciated that a single aperture, or any desired number of apertures such as two, three, four, or more, can be provided in a given embodiment to suit the purpose to which the device 100 is intended to be used with. The distal region 110 of the vent device has a distal end edge 155, or tip-most end. The tip most-end 155 can be solid, or at least not have any aperture for inlet gas flow. As shown, the plurality of apertures 114 of the inlet are located proximally of the distal end edge 155. In a particular example, at least one aperture 114 is provided at a side edge of the distal region and optionally at least one aperture is provided at a side edge of the intermediate region. The side edges of the distal region and the intermediate region can be angled to the distal end edge 155. In a further example, at least one aperture 114 is provided at each of the two side edges of the distal region, each of the two side edges being angled to the distal end edge. In another example, at least one aperture 114 is provided at each of the two side edges of the intermediate region, each of the two side edges being angled to the distal end edge. When two or more apertures 114 are present, the two or more apertures can have the same opening size or different sizes, including the same opening type or different opening types.

The proximal region 112 (FIG. 1) of the housing 108 forms the part of the housing 108 that is in communication with the surrounding, or ambient, environment during use. For example, the proximal region 112 can be exposed or open to the ambient atmosphere for exhausting gas removed through the device directly out into the ambient atmosphere. Thus, gas escaping from the outlet 104 in the proximal region 112 can be vented to atmosphere.

FIG. 1 further shows an outlet 104 configured as a plurality of apertures 116 only some of which, for clarity of FIG. 1, are provided with reference numerals. It will be appreciated that a single aperture, or any desired number of apertures such as two, three, four, six, or more, can be provided in a given embodiment to suit the purpose to which the device is intended to be put. The proximal region 112 of the vent device has a proximal end edge 153, or rear-most end. The proximal end edge 153 has the outlet 104 located therein for outlet gas flow. In an example, at least two or more spaced apart apertures 116 are provided as the outlet for gas outflow. When two or more apertures 116 are present at the proximal region, the two or more apertures can have the same opening size or different sizes, including the same opening type or different opening types. Each of the at least two or more spaced apertures 116 at the proximal region can have an opening defining a plane and wherein the plane is angled to the plane of the aperture opening at the distal region. Consequently, the flow path internally of the housing 108 of the vent device of the present invention is non-linear in that gas flow through the housing and into the flow path must make one or more turns between entering through the inlet and exiting out the outlet.

From the views of FIGS. 1 and 2, the housing 108 is shown having an inlet 102 at the distal region 110 and an outlet 104 at the proximal region, which form the inlet and outlet respectively of the flow path 106, which is formed internally of the housing 108. Both the inlet and the outlet can have one or more apertures and the one or more apertures can straddle a seam 105 extending through the housing.

Thus, an aspect of the prevent invention is understood to include a vent device configured to be used with and to remove gas from a medical device, such as from a Luer adapter, an IVC, a PIVC, etc. The vent device can be configured to remove gas without having to actuate the vent device or move any internal components of the vent device to allow venting. In its simplest form, the vent device only has to be placed in fluid communication with the medical device having gas to be removed without having to actuate or cause any internal parts of the vent device to actuate, such as causing one part of the vent device to move relative to another part of the vent device.

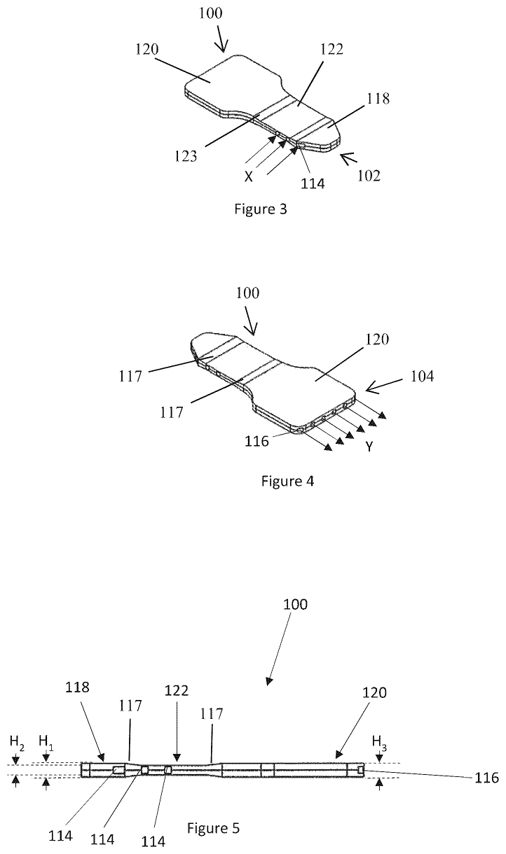

While the housing 108 has a distal region 110 and a proximal region 112, some embodiments of the vent device in accordance with aspects of the present invention do not include a housing 108. For example, and as further discussed below, FIG. 12 shows an alternative vent device 100 that has a distal region and a proximal region and permits gas to flow through or across the device without a separate housing, as further discussed below. Thus, without a housing 108 and with reference to FIG. 3, a vent device 100 can have a distal region 118, a proximal region 120 and an intermediate region 122 having a valve contact area 123 located intermediate the distal region 118 and proximal region 120. Thus, the vent device 100 can embody an insert to be located within a housing 108 or the vent device can have its own utility without being located within a housing. The vent device is therefore usable with a connector from which to remove gas or to vent gas without a separate housing. The vent device can alternatively be formed as an insert to be located within a housing 108 and the combination insert and housing used to remove gas from a line of a medical device.

The distal region 118 comprises an inlet 102, presently formed as a plurality of apertures 114. In some embodiments, the entirety of the inlet 102 may be formed in the distal region 118 of the device 100 and, in other embodiments such as that shown, the inlet 102 may be partially provided on or in the intermediate region 122. In other words, a plurality of apertures that function as an inlet can be in the distal region and in the intermediate region of the device.

As shown in FIG. 4, the proximal region 120 of the device comprises the outlet 104, presently configured as a plurality of apertures 116. The apertures can be spread or located in spaced apart relations at the proximal edge of the proximal region. One or more apertures can optionally be provided at the corner of the proximal edge and the side edge of the proximal region. In still other examples, one or more apertures can be provided at the side edge of the proximal region.

For illustration purposes, FIGS. 3 and 4 are provided with arrows to indicate the directions of inflow and outflow of gas through the device 100. In particular, FIG. 3 shows the inflow of gas, such as from a line of a medical device, as indicated by arrows X pointing into aperture 114 of inlet 102. Similarly, FIG. 4 shows the outflow or venting of gas from the device 100 and thereby from the line, as indicated by arrows Y extending from apertures 116 of outlet 104. Thus, gas from a line, for example, can vent through the vent device 100 by flowing into the inlet 102, through one or more flow paths inside the body of the device, and then out the outlet 104.

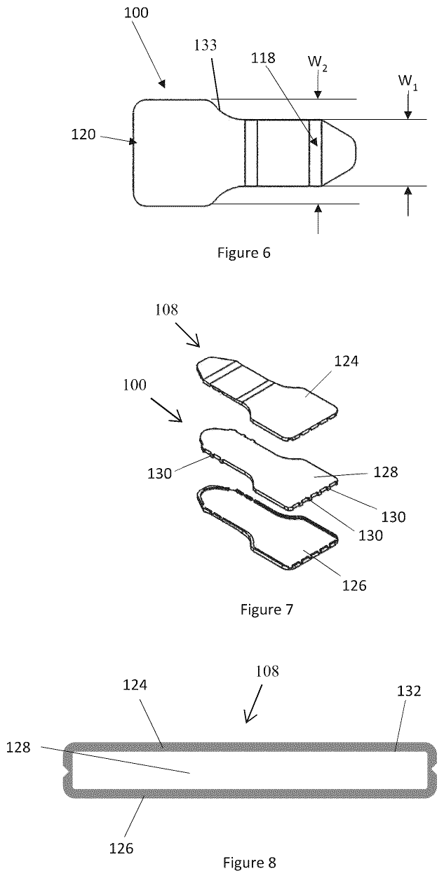

As shown in FIGS. 5 and 6, the distal region 118 of the present embodiment has a first thickness H₁. The intermediate region 122 has a second thickness H₂. H₂ is smaller than H₁. Similarly, in the present embodiment, the proximal region 120 has a third thickness H₃ that is greater than H₂. In some cases, H₁ and H₃ may have the same thickness or different thicknesses. In other cases, H₃ may have the same thickness as H₂. In some examples, H₁ equals H₂ or H₁ is smaller than H₂. Where these alternative thicknesses are incorporated, the distal region is provided with alternative structures for resisting removal of the device from a port or a connector. For example, the distal region can include a strip or a bump to increase the thickness of the distal region at specific locations of the distal region.

Transition sections 117 are provided between two adjacent regions, such as between the distal region and the intermediate region, and also between the intermediate region and the proximal region. The transition sections 117 can embody tapered thickness sections of the vent device and can be provided on just one side of the device or on both sides of the device, as shown in the side elevation view of FIG. 5.

In an example, the vent device 100 can be incorporated with a housing of a connector and the combination housing and the vent device can connect to a port having the line with gas to be removed, such as shown in FIGS. 14-17. In an alternative embodiment, the port with the line having gas to be removed can have a receiving flap with an opening that can receive the vent device 100 directly. Thus, a vent device in accordance with aspects of the present invention can connect directly to a port of an adapter of a medical device to remove gas from the medical device without having to first be assembled to a connector with a tip to then insert the tip into the port of the adapter of the medical device.

In an example, the connector that grips the device 100 so that the combination can then be used with a port can be a resilient member which, for illustration purposes only, will be described as a rubber septum. The rubber septum of the connector can be sized and shaped to grip the intermediate region 122 of the vent device 100. Thus, by enlarging the distal region 118, such as by making the thickness H₁ of the distal region greater than the thickness H₂ of the intermediate region, the device 100 is less likely to inadvertently slip away from the rubber septum to separate from the line. The thicker H₁ distal region creates an interference or restriction point with the rubber septum should the device 100 inadvertently slides proximally out of the connector. In this way, the opening of the rubber septum provides a clamp-like feature with a gap and the relatively thicker distal region of the device 100 is restricted from escaping by the smaller gap of the rubber septum in the proximal direction.

To remove the device 100 from the line, the rubber septum should be compressed along a lateral direction of the receiving end to increase the size of the gap of the receiving end to then permit passage of the distal region 118 out away from the rubber septum. In an example, the receiving end of the connector or port with gas to be vented has a generally elongated opening or a slotted opening. Thus, to open the receiving end of the connector or port, the two short sides of the opening or slot are squeezed together to open the gap of the elongated or slotted opening. In other examples, the rubber septum has a first hardness value and the vent device has a second hardness value that is harder than the first hardness value and wherein when the vent device is gripped and retracted to remove from the rectangular opening, the rubber septum is deformed by the vent device to then allow the vent device to escape from the rubber septum.

The proximal region 120 can have any appropriate thickness. It is enlarged in the present embodiment when compared with the thickness of the intermediate region 122 to afford easy gripping by a practitioner to facilitate removal of the device 100 from the connector and thereby from the line, when desired. However, the thickness of the proximal region can be less than the thickness of the intermediate region and still enable gripping and pulling by the practitioner. In still other examples, the proximal region can be equipped with an enlarged gripping block or surface to facilitate gripping. For example, a strip of material, such as hydrophilic material, can be attached to the proximal end to provide an enlarged surface for gripping.

With reference to FIG. 6, the distal region 118 has a first width W₁ and the proximal region 120 has a second width W₂. W₂ is greater than W₁. The device 100 also has a variable width region 133 between the proximal and distal regions. In some examples, the variable width region 133 is instead an abrupt or single angle riser in transitioning between the smaller width to the larger width. While some parts or sections of the device 100 can have a constant width, the distal most end of the distal region 118 can have a width with a narrowing profile or tapering profile, such as a width variation, to ensure fit and facilitate insertion of the tip into the port or connector to access the line with gas to be removed. The proximal region 120 can be sufficiently large to facilitate easy gripping and removal of the device 100 from the line when desired.

The inlet 102 and the outlet 104 defined opposite ends of the flow path 106, as previously discussed. Moreover, the flow path 106 is configured to vent gas from the interior of the line having gas to be removed out the outlet 104 but not the flow of liquid from the interior of the line out the outlet 104. In some embodiments, restricting liquid flow through the vent device can be achieved by placing a hydrophilic material in the housing 108 that, upon swelling, closes a valve diaphragm or similar flow passages. Thus, upon liquid penetrating the housing 108, the hydrophilic material will swell and prevent liquid from escaping the housing. Alternatively, in the embodiments shown, the flow path may comprise a gas-permeable, hydrophobic material.

The term “gas permeable” in the present context will generally refer to “air permeable”. The term is intended to refer to materials through which can pass gases of the nature of those that become trapped in medical lines. Moreover, the term “hydrophobic” is generally intended to refer to blood repellent, so as to inhibit or prevent the passage of blood out the outlet 104 when the device 100 is in use but permits the passage of gas.

With reference to FIG. 7, the vent device 100 comprises a housing 108, which may be formed in two parts 124, 126, and an insert 128 made of a hydrophobic material sitting in a cavity of the housing on assembling of the two parts 124, 126 together. The two housing parts 124, 126 have cut-outs or surface features that upon assembling the two housing parts form the noted inlet 102 and outlet 104, each with one or more apertures. For example, the upper housing part 124 has a half-circle or half-rectangular cut-out and the lower housing part 126 has a complementary half-circle or half-rectangular cut-out and wherein when the upper and lower housing parts 124, 126 are assembled, the two half cut-outs join to form an aperture with a circumference. Each housing part can comprise more than one cut-out at the inlet and at the outlet for forming more than one aperture at the inlet and at the outlet.

The hydrophobic material insert 128 can be located in the cavity of the two-part housing 108 and the two housing parts secured together, such as by welding, adhesive, detents, or combinations thereof. The insert 128 can be disposed between the inlet 102 and the outlet 104 of the housing, such as serving as a physical presence between the inlet and the outlet. The insert 128 can be provided with a plurality of projections 130 alongside edges thereof to occupy the apertures 114 of the inlet 102 and the apertures 116 of the outlet 104. In other examples, the projections 130 can be omitted and the outer perimeter of insert 128 held tightly against the side edges of the housing 108 in the assembled state, such as with some interference or compression. In still other examples, the insert can have a plurality of projections 130 as well as be held tightly against the side edges of the housing when assembled within the housing. To maintain clarity of the drawings, only some of projections 130 are labelled.

In an example, each of the two housing parts 124, 126 has a housing contour that is complementary to the contour of the insert 128. In other examples, the insert can have a different contour than the contour of the two housing parts. For example, the insert made from a hydrophobic material can embody a first insert section that occupies the distal region cavity of the housing and covering the inlet at the distal region and the intermediate region. A separate second insert section occupies the proximal region of the housing and covering the outlet at the proximal region. In an example, the first insert section can be spaced from the second insert section. As shown, with reference to FIGS. 6 and 7, the vent device 100 can have a body with a generally flat profile such that the length of the body is at least 20 times (20×) the thickness of the body. The length can be greater than the thickness of the body by about 25×, such as greater than 30×, 35×, 50×, or more. The large length to thickness ratio defines a thin profile for inserting into a slotted or elongated opening, as opposed to a round opening, of a port or a connector.

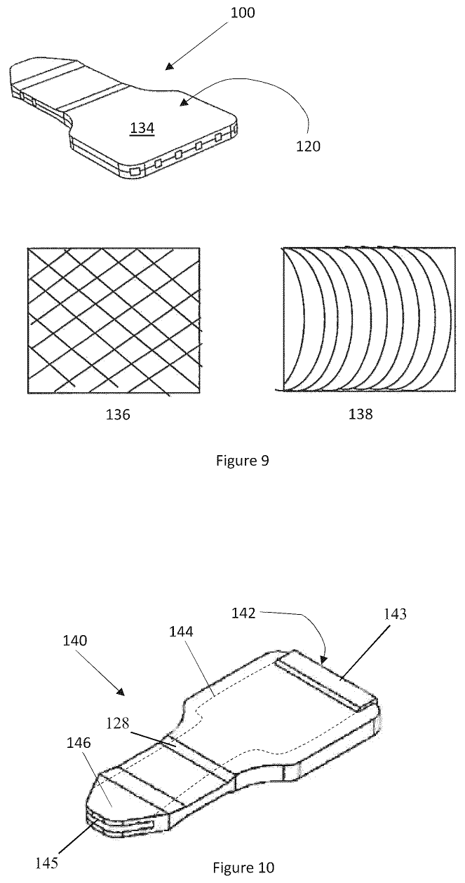

FIG. 8 is a cross-sectional view of the assembled housing 108 showing the insert 128 of hydrophobic, gas-permeable, material occupying substantially the entirety of the internal cavity 132 formed by the two parts 124, 126 of the housing 108.

To remove the device 100 from the line from which gas is to be removed, a small amount of force is required to pull the distal region of the device 100 through the connector or valve. To assist with application of this force to pull the device 100, the proximal region 120 of the device is configured as a grip, as shown in FIG. 9. This configuration may be achieved by making the proximal region 120 sufficiently large or otherwise shaped to afford easy gripping. In an example, the external surface 134 of the device at the proximal region 120 may be textured. Two of many possible variations of texture 136, 138 are illustrated that can be incorporated on the external surface 134 of the device 100. In some examples, external surfaces 134 on both sides of the device can be textured.

An alternative vent device 140 is shown in FIG. 10, in which the outlet 142 comprises the hydrophobic material. As stated above, the outlet 142 defines an end of the flow path 144, which has been illustrated in broken lines as being internal of the device 140. Thus, by incorporating or using a hydrophobic material at the outlet 142, the flow path 144, in part defined by the outlet, is configured to inhibit the flow of liquid.

The housing 146 of the device 140 comprises one or more proximal outlet apertures (not shown), like those used for the outlet 104 of device 100 discussed elsewhere herein. However, in the present embodiment, the outlet 142 is provided with a strip 143 of a hydrophobic material placed across the one or more outlet apertures. The strip 143 thus covers the one or more outlet apertures. The flow path 144 internally of the housing 146 may also comprise an insert 128 made of a hydrophobic material or may be a hollow cavity or conduit within the housing without any strip. When incorporating a strip 143 of hydrophobic material with the housing and covering the one or more outlet apertures on the housing, a distal opening 145 can be provided at the distal region. The distal opening 145 can be an elongated slot that follows the varying width of the distal region. The distal opening can be formed at the distal end edge of the distal region.

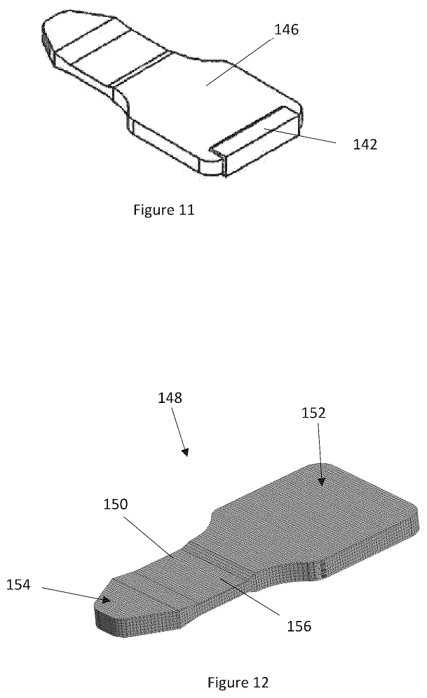

FIG. 11 shows the device 140 from a rear perspective view, with the strip 142 attached to the housing and covering the outlet, such as by adhesion, ultrasonic welding or any other suitable process. In an example, another hydrophobic material strip may also be attached to the distal region of the housing 146, to cover the inlet at the distal region. In an alternative embodiment, the distal region of the vent device 140, which can embody an insert, to be placed inside the housing 146 can include a hydrophobic material to restrict liquid flow at the inlet but the distal region of the housing 146 itself is not provided with a strip, unlike the proximal region.

The flow path of the device 100 can be formed by, or is substantially coincident with, a body of a hydrophobic material. For example, the flow path can comprise the cavity of the housing occupied in its entirety by the hydrophobic material insert and gas flows across the material of the insert.

FIG. 12 shows a further embodiment in which the device 148 itself is formed by a body 150 of hydrophobic material. The vent device 148 of FIG. 12 is usable to vent gas from a line of a medical device without first inserting the body of the device into a complementary housing. In the present embodiment, the outlet is defined by a proximal region 152 of the body 150 and the inlet is defined by a distal region 154 of the body 150. Thus, wherever gas from the line having gas to be removed enters the body 150 of the insert, the inflow of gas in the distal region 154, wherever gas enters, will be considered an inlet, and wherever that gas escapes the body 150 into the surrounding environment will be considered an outlet. In an example, the distal region 154 of the vent device 148 is inserted into a receiving end of a port or adapter of a medical device having gas to be removed and gas can foreseeably enter all exposed surface areas of the insert located within the port or adapter. In that particular example, the entirety of the distal region of the insert that gas enters can be considered the inlet.

While the distal region 154 may extend all the way back to the proximal region 152, for the purpose of illustration, the device 148 will be considered to include the distal region 154, which again at least partially comprises the inlet, the proximal region 152 comprising the outlet, and an intermediate region 156 intermediate the distal region 154 and proximal region 152.

The same comments as set out above, relating to the relative thickness and width of the various regions of the device 100 apply similarly to the device 148 of the present embodiment, which can have varying thicknesses and is issuable without first being inserted into a complementary housing.

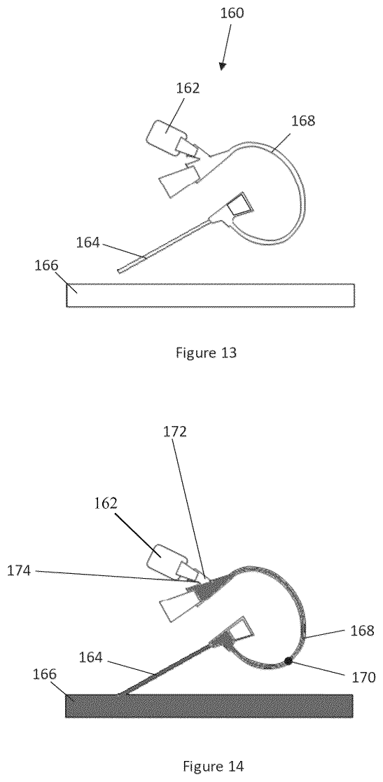

FIG. 13 shows a prior art peripheral intravenous catheter (PIVC) access assembly 160 with a needleless connector 162, prior to insertion of a needle 164, having a catheter tube located thereon, into the vasculature 166 of a patient. The tubing line 168 of the assembly 160 between the side port of the catheter hub and the adapter is empty or free of blood prior to use. FIG. 14 shows the same assembly as FIG. 13 but after insertion of the needle 164 into the vasculature. Blood 170 is shown progressed up the needle 164 and into the tubing 168. In particular, an air pocket 172 is formed at the junction 174 of the adapter, including in the needleless connector 162 by the advancing blood. Normally, a practitioner must insert a Luer tip of a medical instrument, such as a syringe, into the needleless connector 162 and extract the air to remove the air pocket 172 and to prevent the air pocket 172 from being injected as a bubble into the vasculature 166 thereby causing an air embolism.

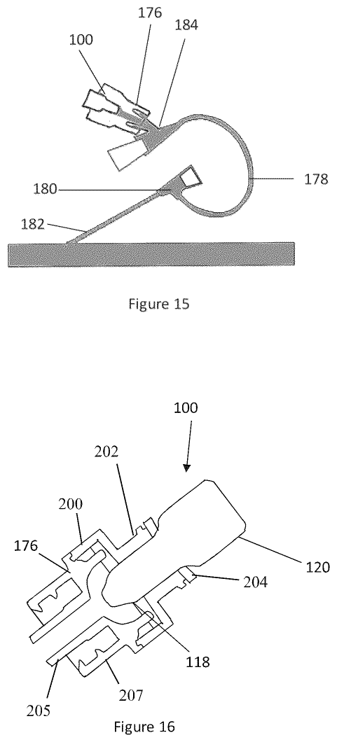

FIG. 15 illustrates a vent device 100 in accordance with present teachings inserted through a needleless connector 176 such that it is in communication with an interior of the line 178. Notably, blood 180 that has progressed up the needle 182, into the line 178, and into the junction 184 will displace or push any air from the junction 184 out through the vent device 100 but blood does not progress through the device 100 due to the flow path of the device being configured to prevent blood from escaping through the outlet of the device 100. For example, and as previously discussed, the device 100 itself may be made from a hydrophobic material or one or more strips made from a hydrophobic material may be placed at the inlet of an insert, at an inlet of a housing having the insert located herein, at an outlet of the housing having the insert, or combinations thereof, to allow gas to pass through the device but not blood.

FIG. 16 is a schematic view of a vent device 100 in accordance with further aspects of the invention inserted into a housing 200 of a connector 176 having a Luer tip 205 and a threaded collar 207 surrounding the tip 205. In some examples, the collar can be omitted and the device is referred to as a Luer slip. The Luer tip 205 of the connector 176 is usable with connectors having a female Luer, such a needleless valve with a female Luer receiving end.

As shown, the housing 200 of the connector 176 has a receiving end 202 comprising a flap or valve 204 having a slit or an opening sized and shaped for receiving the distal region 118 of the vent device 100. The receiving end 202 can receive the distal region 118 and at least part of the intermediate region of the device 100, but not the proximal region 120 of the device 100, which extends outside of the housing 200. Thus, by making the proximal region 120 too wide, and too long, to fit into the connector 176, the proximal region 120 precludes inadvertent pushing of the entirety of the device 100 through the housing 200 and into the junction 184 of the adapter. Further, the transition region having a variable width or an abrupt change in width between the intermediate region and the proximal region can be sized and shaped to preclude insertion into the receiving end 202 of the housing 200.

FIG. 17 shows the same device 100 and needleless connector 176 as that of FIG. 16 but from a perspective that has rotated 90° from that of FIG. 16, to show the thickness of the device 100 instead of the width of the device. As shown, the intermediate region 122 is held by the perimeter of the opening of the flap 204 of the connector 176. The thicker distal region 118 is further situated within the housing 200 of the connector 176 and in communication with the interior of a line in the direction indicated by arrow Z.

Methods of using and of making vent devices and vascular access assemblies, including components of vascular access assemblies, as shown and described herein are understood to be within the scope of the present invention.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Although limited embodiments of vent devices, connectors, and vascular access assemblies and their components have been specifically described and illustrated herein, many modifications and variations will be apparent to those skilled in the art. Accordingly, it is to be understood that the vent devices, connectors, and vascular access assemblies and their components constructed according to principles of the disclosed devices, systems, and methods may be embodied other than as specifically described herein. The disclosure is also defined in the following claims.

1. A vent device for venting gas from a medical device, the vent device comprising:
an inlet in communication, in use, with an interior of the medical device; and
an outlet open to air outside of the medical device;
wherein the inlet and the outlet define opposite ends of a flow path configured to vent gas from the interior of the medical device out the outlet and inhibit flow of liquid from the interior of the medical device out the outlet.

2. The vent device of claim 1, wherein the flow path comprises a gas-permeable hydrophobic material.

3. The vent device of claim 1, further comprising a housing, the inlet being at a distal region of the housing, the outlet being at a proximal region of the housing, and the flow path forming a conduit within the housing.

4. The vent device of claim 3, wherein the hydrophobic material is located at the outlet.

5. The vent device of claim 4, wherein the housing comprises one or more proximal outlet apertures and the hydrophobic material is located at the outlet or a strip of hydrophobic material is positioned across the one or more outlet apertures.

6. The vent device of claim 5, wherein the strip of hydrophobic material covers the one or more outlet apertures such that gas flowing out of the one or more apertures must flow through the strip of hydrophobic material.

7. The vent device of claim 3, wherein the hydrophobic material is formed as an insert and the insert is located in a cavity of the housing and disposed between the inlet and outlet.

8. The vent device of claim 3, wherein the inlet comprises one or more inlet apertures formed in the distal region of the housing.

9. The vent device of claim 2, wherein the flow path is formed by a body of the hydrophobic material.

10. The vent device of claim 8, wherein the vent device has a generally flat profile with a length and a thickness, and wherein the length is at least 20 times greater than that of the thickness.

11. The vent device of claim 1, wherein the vent device comprises a distal region at least partially comprising the inlet, a proximal region comprising the outlet and an intermediate region intermediate the distal region and proximal region.

12. The vent device of claim 11, wherein the distal region has a first thickness and the intermediate region has a second thickness that is smaller than the first thickness.

13. The vent device of claim 11, wherein the proximal region has a third thickness that is greater than the second thickness.

14. The vent device of claim 11, wherein the proximal region is configured as a grip.

15. The vent device of claim 14, wherein an external surface of the proximal region is textured.

16. The vent device of claim 11, wherein the distal region has a first width and the proximal region has a second width that is greater than the first width.

17. The vent device of claim 1, wherein the line is part of an intravenous catheter (IVC) or peripheral intravenous catheter (PIVC).

18. The vent device of claim 17, wherein the inlet is located within a receiving end of a port or a connector.

19. The vent device of claim 1, wherein gas vents through the vent device without actuating a first part of the vent device relative to a second part of the vent device.

20. A method of forming a vent device for venting gas from a line having gas to be removed, said method comprising:
forming a body from a hydrophobic material with a distal region, a proximal region, and intermediate region between the distal region and the proximal region;
providing the body with a length that is at least 20 times greater than a thickness of the body to form a thin profile;
forming the distal region with a first width W₁ and the proximal region with a second width W₂ with the second width W₂ being greater than the first width W₁;
forming the distal region with a first thickness H₁, the intermediate region with a second thickness H₂, and the proximal region with a third thickness H₃; and
providing a tapered edge at the distal region to facilitate insertion of the vent device.

21. The method of claim 20, wherein the first thickness H₁ is greater than the second thickness H₂.
